(19) United States
(12) Patent Application Publication
(10) Pub. No.: US 2026/0053754 A1
(43) Pub. Date: Feb. 26, 2026

(54) KAEMPFEROL BIOMIMETIC NANO-MATERIAL, METHOD FOR PREPARING SAME AND USE THEREOF

(71) Applicant: NANJING DRUM TOWER HOSPITAL, Nanjing (CN)

(72) Inventors: Jinglin WANG, Nanjing (CN); Haozhen Ren, Nanjing (CN)

(73) Assignee: NANJING DRUM TOWER HOSPITAL, Nanjing (CN)

(21) Appl. No.: 19/263,565

(22) Filed: Jul. 9, 2025

Related U.S. Application Data
(63) Continuation of application No. PCT/CN2024/114964, filed on Aug. 28, 2024.

(30) Foreign Application Priority Data
Aug. 20, 2024 (CN) 202411148209.2

(51) Int. Cl. A61K 9/51; A61K 9/1271; A61K 9/1277; A61K 31/352; A61K 47/02; A61K 47/18; A61K 47/20

(52) U.S. Cl. CPC A61K 9/5176; A61K 9/1271; A61K 9/1277; A61K 9/5192; A61K 31/352; A61K 47/02; A61K 47/18; A61K 47/20

(57) ABSTRACT

A kaempferol-mesenchymal stem cell membrane biomimetic nano-material allowing for more accurate targeting to injured issues is provided. The biomimetic nano-material is simple in preparation method, high in universality, and suitable for large-scale production, mesenchymal stem cell membranes are used to improve the biocompatibility and targeting ability of carriers and increase the in vivo circulation time of drugs; the biomimetic nano-material allows for targeted delivery of kaempferol to the liver, providing a novel treatment method for ALF.

### CROSS REFERENCE TO THE RELATED APPLICATIONS

This application is a continuation application of International Application No. PCT/CN2024/114964, filed on Aug. 28, 2024, which is based upon and claims priority to Chinese Patent Application No. 202411148209.2, filed on Aug. 20, 2024, the entire contents of which are incorporated herein by reference.

### TECHNICAL FIELD

The present invention belongs to the technical field of biomedical materials, and in particular relates to a kaempferol biomimetic nano-material, a method for preparing the same and a use thereof.

### BACKGROUND

Acute liver failure (ALF) refers to a clinical syndrome occurring without underlying liver diseases and with liver functions deteriorating rapidly in a short time to cause ascites, coagulation disorders, hepatic encephalopathy, and multi-organ failure. There are a variety of causes of ALF, with the main causes including acetaminophen toxicity, drug-induced liver injury, viral infection, ischemia, and autoimmune diseases. The causes of ALF vary worldwide. ALF caused by N-acetyl-para-aminophenol (APAP) overdose is common in Europe and other developed countries, while ALF caused by viral hepatitis (types A, B and E) occurs mainly in developing countries. ALF patients have poor prognosis, and there is no effective therapy. Hence, how to treat ALF has become a key challenge.

Kaempferol, also known as kaempferol-3, kaempferide, or kaempherol, is a dietary flavonoid compound mainly derived from the rhizome of Kaempferia galanga. As a very common compound in nature, kaempferol is widely found in various fruits, vegetables and natural plants. Kaempferol acts on a large amount of signal targets in vivo, and has extensive pharmacological activities such as anti-tumor, anti-inflammation, anti-oxidation, cardio-protection and hepatoprotection. However, it has poor water solubility and shows a first-pass effect on gastrointestinal tract in vivo, which largely limits its clinical application.

Liposomes are spherical nano-shells resulting from the spontaneous closure of phospholipid molecules in an aqueous solution. They may entrap water-soluble molecules in lumens and liposoluble molecules between phospholipid bilayers at the same time to improve the bioavailability of water-insoluble drugs, exhibiting a high drug loading and sustained-release property. However, they have challenges such as rapid clearance by the mononuclear phagocyte system (MPS) and lack of selectivity between healthy and injured liver cells.

In recent years, natural cells such as erythrocytes, 12 dendritic cells (DCs), 13 mesenchymal stem cells (MSCs) and 14 macrophagocytes have been explored as drug carriers. Mesenchymal stem cells (MSCs) are pluripotent progenitor cells with self-renewal and pluripotent differentiation potential. The application of stem cell therapies is gradually deepened and diversified, showing broad prospects. MSCs may regulate the activation and proliferation of T cells, regulate cytokines produced by immune cells, and specifically target the site of injury to thus inhibit inflammation and carry out immune regulation. MSC membranes share the advantages of active targeting ability, immune escape characteristics and long cyclicity of MSCs, endowing the MSC membranes with the prospect of targeted drug delivery due to their inherent targeting ability and low immunogenicity. However, some investigators have demonstrated that MSCs may promote tumor progression and even differentiate into tumors ((F. Ma et al., Human Umbilical Cord Mesenchymal Stem Cells Promote Breast Cancer Metastasis by Interleukin-8- and Interleukin-6-Dependent Induction of CD44+/CD24− Cells. Cell Transplantation 24, 2585-2599 (2015).). In addition, some alarming results show that whole-cell drug carriers may be contaminated by biomaterials and raise biosafety concerns. In order to overcome these defects, it has been reported that new cell membrane-coated particles with a minimal loss of membrane proteins have been developed (A. V. Kroll, R. H. Fang, L. Zhang, Biointerfacing and Applications of Cell Membrane-Coated Nanoparticles. Bioconjugate Chemistry 28, 23-32 (2016).). At present, MSC membrane-modified NPs have made gratifying progress in anti-inflammation, tissue regeneration and tumor treatment (L. Fan, A. Wei, Z. Gao, X. Mu, Current progress of mesenchymal stem cell membrane-camouflaged nanoparticles for targeted therapy. Biomedicine & Pharmacotherapy 161, (2023).).

### SUMMARY

Object of the present invention: An object of the present invention is to provide a kaempferol-mesenchymal stem cell membrane biomimetic nano-material allowing for more accurate targeting to injured issues. The biomimetic nano-material is simple in preparation method, high in universality, and suitable for large-scale production.

### The Technical Solution

The present invention provides a kaempferol biomimetic nano-material, including liposomes for mesenchymal stem cell membranes and kaempferol liposomes. The nano-material has an average particle size of 135-140 nm.

The present invention further provides a method for preparing the kaempferol biomimetic nano-material includes the steps of:
(1) preparing the mesenchymal stem cell membranes: culturing and collecting the mesenchymal stem cells, sequentially performing centrifuging, first resuspending, centrifuging, second resuspending, freezing-thawing, and centrifuging on the cells, and finally collecting cell pellets, which are the mesenchymal stem cell membranes;
(2) preparing the kaempferol liposomes: dissolving lecithin, cholesterol and kaempferol in absolute ethanol to obtain a mixture, adding the resulting mixture to ultrapure water, stirring the mixture, and extruding the mixture using a liposome extruder to pass through films to obtain the kaempferol liposomes; and
(3) extruding the mesenchymal stem cell membranes, obtained in step (1), using the liposome extruder, mixing the extruded mesenchymal stem cell membranes with kaempferol liposomes, ultrasonically shaking a resulting mixture in an ice bath, and again, extruding the mixture using the liposome extruder to obtain the kaempferol biomimetic nano-material.

Further, in step (1), the first resuspending is to resuspend the cells in a phosphate buffer saline, and the second resuspending is to resuspend the cells in a hypotonic solution.

Further, the hypotonic solution is an aqueous solution containing 10 mmol/L tris (hydroxymethyl)-aminomethane hydrochloride (Tris-HCL), 1 mmol/L KCl, 1.5 mmol/L MgCl2, and 1 mmol/L phenylmethanesulfonyl fluoride (PMSF) by concentration content, respectively.

Further, in step (2), a mass ratio of the lecithin to the cholesterol to the kaempferol is 3:1:1.

Further, in step (3), a mass ratio of the extruded mesenchymal stem cell membranes to the kaempferol liposomes is 1:1-1:10.

Further, in step (1), the centrifuging is carried out at 4° C. in each case.

Further, in step (2), the kaempferol liposomes are obtained by sequential extruding through polycarbonate films of 800 nm, 400 nm and 200 nm on the liposome extruder.

The present invention further provides a use of the kaempferol biomimetic nano-material in preparation of a drug for treatment of acute hepatic failure.

### Beneficial Effects

(1) The biomimetic nano-material provided by the present invention is obtained by physical extrusion, i.e., film extrusion, and is easy to prepare and easy to produce on a large scale.
(2) The present invention designs a biomimetic nano-material designed, and mesenchymal stem cell membranes are used to improve the biocompatibility and targeting ability of carriers and increase the in vivo circulation time of drugs.
(3) The biomimetic nano-material prepared according to the present invention allows for targeted delivery of kaempferol to the liver, providing a novel treatment method for ALF.

### BRIEF DESCRIPTION OF THE DRAWINGS

FIG. 1 shows a schematic diagram of a process for preparing a biomimetic nano-material KAE@ML;

FIGS. 2A-2E show schematic diagrams of the characterization of the biomimetic nano-material. The diagrams are: (FIG. 2A) transmission electron microscopy (TEM) images of biomimetic nano-materials L and ML, with a scale size of 100 nm; (FIG. 2B) dynamic light scattering (DLS) measured images of the biomimetic nano-materials L and ML; (FIG. 2C) Zeta potentials of the biomimetic nan-materials L and ML; (FIG. 2D) entrapment efficiency and drug loading of M and L at three different ratios; (FIG. 2E) in vitro cumulative drug release of biomimetic nano-materials KAE, KAE@L and KAE@ML in PBS at PH 7.4;

FIG. 3 shows cell viability images of the biomimetic nano-materials KAE, KAE@L and KAE@ML;

FIGS. 4A-4B show the in vivo targeting ability of the biomimetic nano-material: FIG. 4A shows the fluorescence intensity of a control group, a PBS group, an L group, an ML group after 1, 3 and 7 days since injection, as detected by an IndiGO imaging system, and FIG. 4B shows typical ex vivo images of major organs in each group 24 h after injection; and

FIGS. 5A-5C show therapeutic effects of the biomimetic nano-materials: FIG. 5A shows determined ALT and AST levels in mice in the control group, the ALF group and the treatment groups, FIG. 5B shows a survival graph of the mice in the control group, the ALF group and the treatment groups, and FIG. 5C shows HE images of rats in each group.

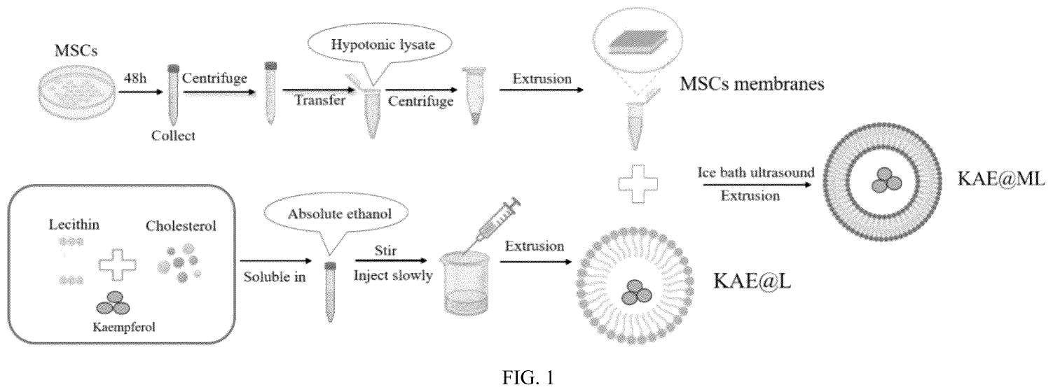

### DETAILED DESCRIPTION OF THE EMBODIMENTS

In order to deepen the understanding of the present invention, the present invention will be further described in detail below in conjunction with embodiments and drawings. The embodiments are only used to explain the present invention and do not constitute a limitation on the scope of the present invention.

The sources of raw materials used in the following examples are as follows:
Mesenchymal stem cells: ATCC Cell Bank of the United State (American Type Culture Collection);
Lecithin: Shanghai Aladdin Biochemical Technology Co., Ltd., 8002-43-5;
Cholesterol: Hubei Dingxintong Pharmaceutical Co., Ltd., D162;
Kaempferol: Shanghai Aladdin Biochemical Technology Co., Ltd., K107144;
Tris-HCL: Shanghai Aladdin Biochemical Technology Co., Ltd., T301502;
KCl: Shanghai Aladdin Biochemical Technology Co., Ltd., P433492;
MgCl2: Shanghai Beyotime Biotechnology Co., Ltd., ST269; and
Phenylmethylsulfonyl fluoride (PMSF): Jiangsu KeyGEN Biotechnology Co., Ltd., KGB5105-10.

### Example 1: Preparation of Kaempferol Biomimetic Nano-Material KAE@ML

### (1) Preparation of Mesenchymal Stem Cell Membranes

(1-1) Mesenchymal stem cells were cultured in a 10 cm large dish according to the prior art (Mesenchymal Stem Cell Membrane-Camouflaged Liposomes for Biomimetic Delivery of Cyclosporine A for Hepatic Ischemia-Reperfusion Injury Prevention), and then collected after the cells grew all over.
(1-2) The cells from step (1-1) were centrifuged at a low speed (1000 rpm, 5 min) at 4° C., cell pellets were collected, resuspended in a pre-cooled phosphate buffer saline (PBS), and centrifuged again (4° C., 1000 rpm, 5 min), and cell pellets were collected. Here, the step of centrifuging was repeated three times.
(1-3) In an ice bath, the cell pellets (about 10⁸ cells) obtained in step (1-2) were suspended in 2 mL of a hypotonic solution (using water as a solvent and including: 10 mmol/L Tris-HCL, 1 mmol/L KCl, 1.5 mmol/L MgCl₂, and 1 mmol/LPMSF) and the cells were resuspended for 6-8 h.
(1-4) In the presence of liquid nitrogen and at room temperature, the cell suspension obtained in step (1-3) was frozen and thawed 5-7 times repeatedly to fully disrupt the cells. The suspension was centrifuged at a low speed (850 g, 15 min) at 4° C., the supernatant was collected and centrifuged (15,000 g, 30 min) again, and the cell pellets, i.e., stem cell membrane debris (M), was fixed in volume with bicinchoninic acid (BCA) and stored at −80° C.

### (2) Preparation of Kaempferol Liposomes

Lecithin, cholesterol and kaempferol were dissolved in absolute ethyl alcohol at a mass ratio of 3:1:1 and ultrasonically shaken; after full dissolution, the mixture was slowly injected into ultrapure water using a 1 ml syringe (over stirring); the mixture was stirred by a magnetic stirrer till no smell of ethanol, and ultrasonically shaken for 5 min at the amplitude of 40% in an ice bath, during which the ultrasonic shaking was performed for 10 s and stopped for 10 s, and so on; and the mixture was sequentially extruded through polycarbonate films of 800 nm, 400 nm, and 200 nm using a liposome extruder, to obtain the kaempferol liposomes (KAE@L).

### (3) Preparation of Daempferol Biomimetic Nano-Material

The mesenchymal stem cell membranes (M) obtained in step (1) were extruded using the liposome extruder of 200 nm and then mixed with the kaempferol liposomes at a volume ratio of 1:1; the mixture was subjected to ultrasonic shaking for 5 min at the amplitude of 40% in an ice bath, during which the ultrasonic shaking was performed for 10 s and stopped for 10 s, and so on; and the mixture was extruded again using the liposome extruder of 200 nm to obtain the nano-material (KAE@ML).

### Comparative Example 2: Preparation of Kaempferol-Free Biomimetic Material ML

This example was the same as Example 1 except that no kaempferol was used in step (2). The nano-material obtained finally was a mixed material ML of the mesenchymal stem cell membranes (M) and the empty liposomes (L).

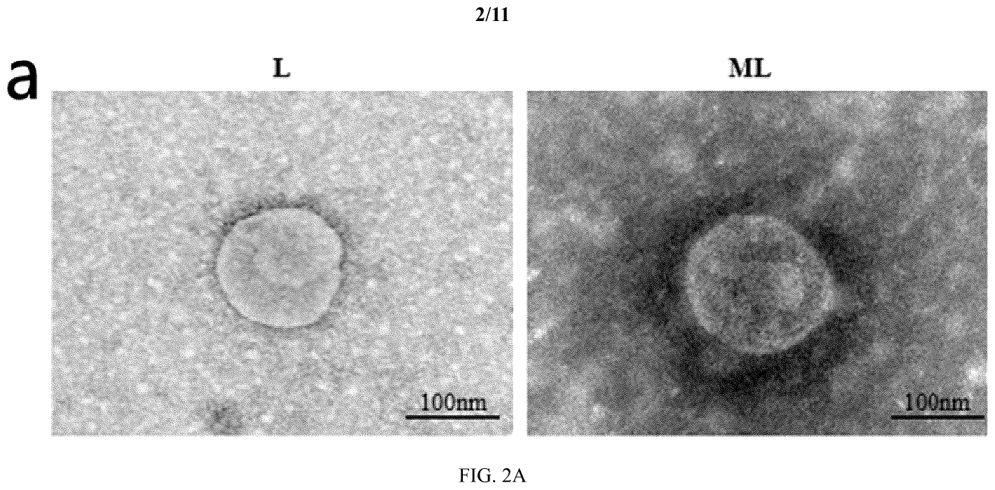

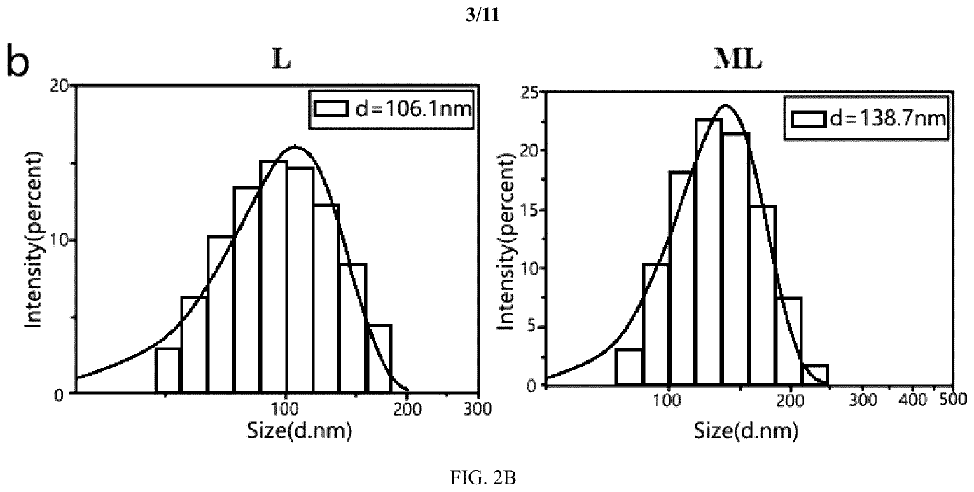

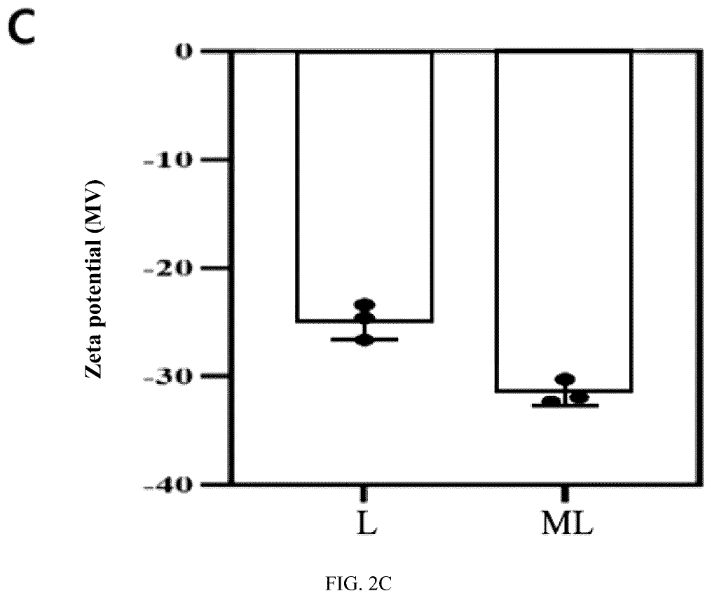

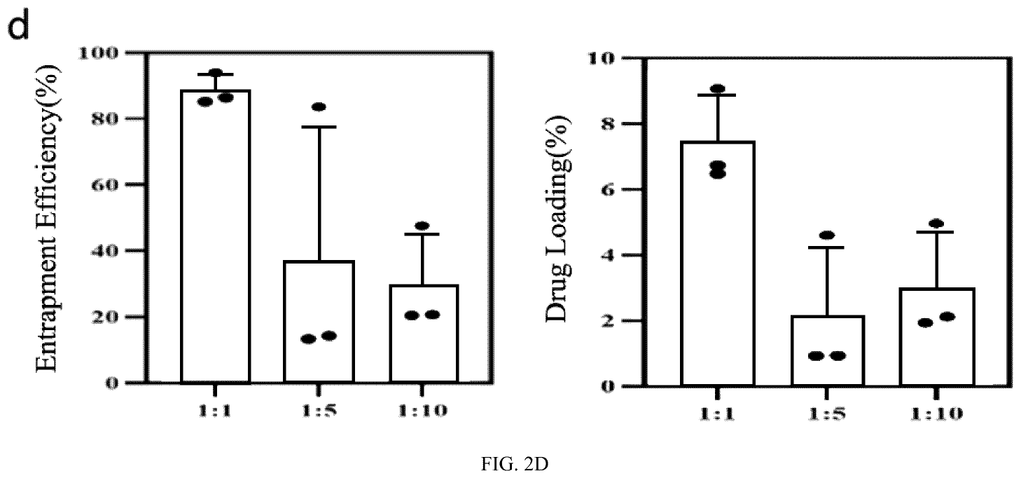

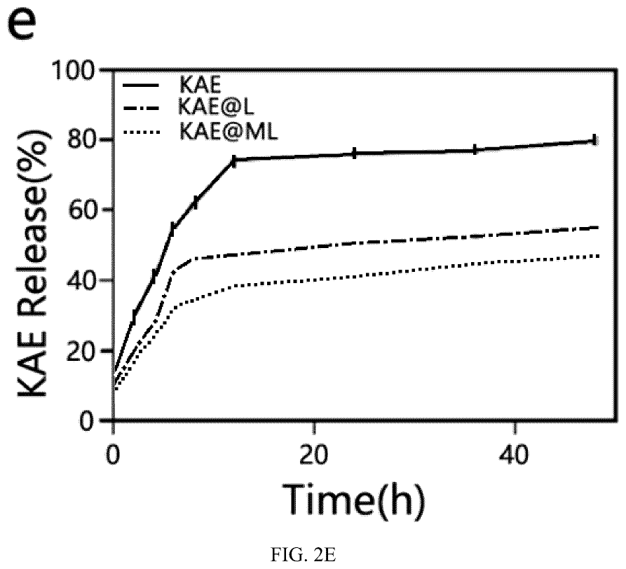

By characterization for the product in Comparative Example 2, the transmission electron microscopy (TEM) images demonstrated the nanoscale structure of the liposome, and the distinctive core-shell structure were observed on the surface of ML (FIG. 2A). Malvern particle sizer testing showed that the average particle sizes of L and ML were 106.1 nm and 138.7 nm, respectively. The particle size of the ML group increased by 32.6 nm compared with that in the L group (FIG. 2B). The average Zeta potentials of L and ML were −24.909 mV and −31.52 mV, respectively, and ML exhibited the negative Zeta potential greater than that of L, indicating that the cell membranes were successfully entrapped on the surfaces of the liposomes (FIG. 2C).

### Example 3: Preparation of Kaempferol-Mesenchymal Stem Cell Membrane Biomimetic Nano-Material KAE@ML

The method in this example was different from Example 1 in that during the preparation of the kaempferol biomimetic nano-material in step (3), the mass ratio of the extruded mesenchymal stem cell membranes (M) to the kaempferol liposomes was 1:10.

### Example 4: Preparation of Kaempferol-Mesenchymal Stem Cell Membrane Biomimetic Nano-Material KAE@ML

The method in this example was different from Example 1 in that during the preparation of the kaempferol biomimetic nano-material in step (3), the mass ratio of the extruded mesenchymal stem cell membranes (M) to the kaempferol liposomes was 1:5.

### Example 5: Entrapment Efficiency and Drug Loading

KAE@MLs obtained in Examples 1, 3 and 4 represented mesenchymal stem cell membranes Ms and kaempferol liposomes KAE@Ls at different mass ratios. As shown in FIG. 2D, Ms and KAE@Ls at different ratios were different in entrapment efficiency and drug loading. When the ratio was 1:1, the KAEs in MLs had the entrapment efficiency of 88.54% and the drug loading of 7.43%; when the ratio was 1:5, the KAEs in MLs had the entrapment efficiency of 37.13% and the drug loading of 2.16%; and when the ratio was 1:10, the KAEs in MLs had the entrapment efficiency of 29.58% and the drug loading of 3.01%. Therefore, when the mass ratio of Ms to KAE@Ls was 1:1, both the entrapment efficiency and the drug loading are high.

### Example 6: In Vitro Drug Release Kinetics

Taking the KAE@MLs prepared in Example 1 as an example, 2%Tween-80/PBS to which KAE showed good dissolubility was used as a drug release medium, the drug content of KAE outside a dialysis bag was detected at fixed time points, and drug release curves were plotted. The results indicated that the cumulative release of free KAE reached 54.88% at 6 h, and 75.37 at about 12 h, after which the release behavior tended to be stable. The release of KAE@Ls was only 42.67% at 6 h, and only 47.33% at 12 h; the release of KAE@MLs reached 32.21% and 38.17% at 6 h and 12 h, respectively (FIG. 2E), indicating that KAE@MLs had a sustained-release effect on a drug and were able to prolong the circulation time of the drug in vivo.

### Example 7: Biocompatibility of Biomimetic Nano-Material KAE@ML

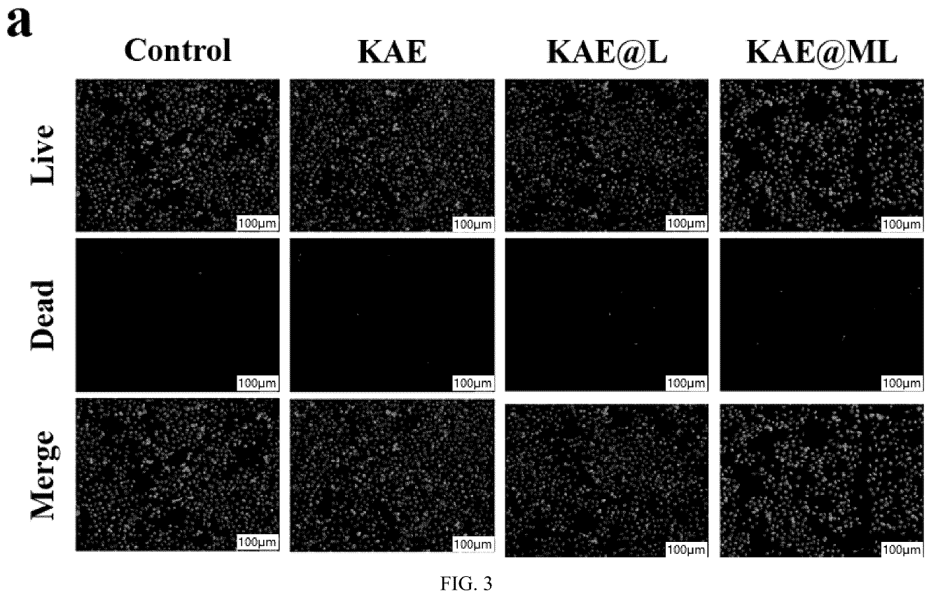

As shown in FIG. 3, kaempferol (KAE), KAE@L, and KAE@ML were co-cultured with liver cells (LO2) for 24 h; the cells were stained, with live cells being green and dead cells being red; and the cell states were observed under a fluorescence microscope (FIG. 3). The results showed that KAE, KAE@L, and KAE@ML showed low toxicity to LO2, exhibiting good biocompatibility.

### Example 8: Targeting Ability of Kaempferol-Free Biomimetic Material ML Prepared in Comparative Example 2

In a control group, normal SD rats were used; in a PBS group, SD rats were injected with free dye Cy5.5 through tail veins; and in an L group, SD rats were injected with Cy5.5-stained blank liposomes through tail veins. In an ML group, SD rats were injected with Cy5.5-stained blank liposomes entrapped with mesenchymal stem cell membranes through tail veins.

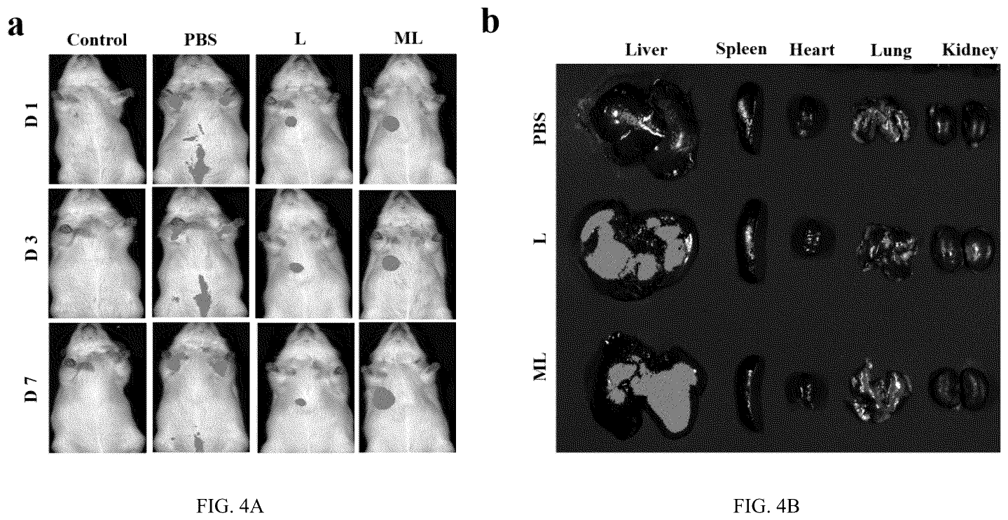

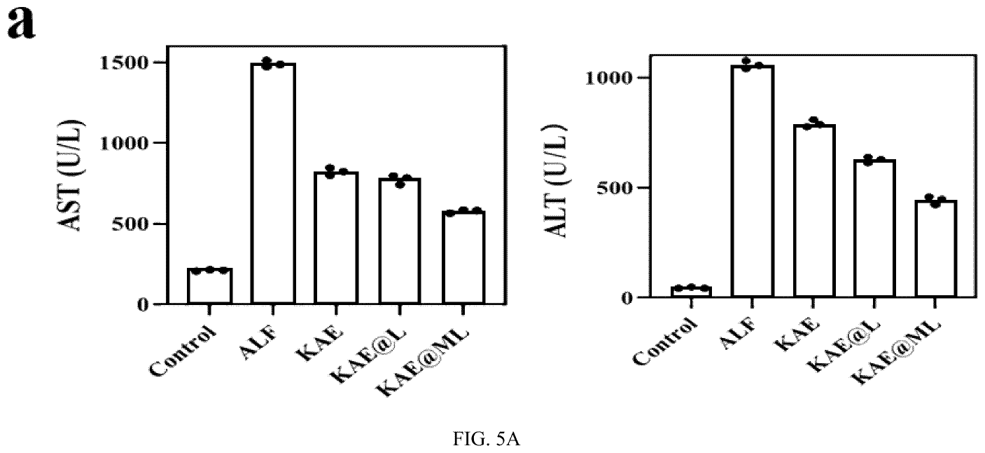

In order to evaluate the in vivo targeting behavior of the biomimetic nano-material, rats were intravenously injected with Cy5.5-loaded biomimetic nano-materials, and the distributions of the biomimetic nano-materials Ls and MLs were monitored at preset time points using the IndiGo imaging system. The results showed that the highest fluorescence intensity of the ML group was reached 24 h after injection. In contrast, almost no fluorescence was observed in the PBS group (FIG. 4A). Then, the rat tissues were dissected, and major organs were imaged to monitor the distribution behaviors of Ls and MLs. The results showed that the fluorescence was mainly distributed in liver tissues, which was consistent with the in vivo imaging results, and the ML groups exhibited the highest fluorescence intensity (FIG. 4B). Therefore, it was demonstrated that the liposomes entrapped with the mesenchymal stem cell membranes (MLs) had good active targeting ability.

### Example 9: Therapeutic Effect of Biomimetic Nano-Materials on Acute Liver Failure

In a control group, normal SD rats were used; in an ALF group, SD rats with ALF were used; in a KAE group, ALF SD rats were intragastrically administrated with kaempferol; in a KAE@L group, ALF SD rats were injected through tail veins with kaempferol liposomes; and in a KAE@ML group, ALF SD rats were injected through tail veins with kaempferol liposomes entrapped with mesenchymal stem cell membranes.

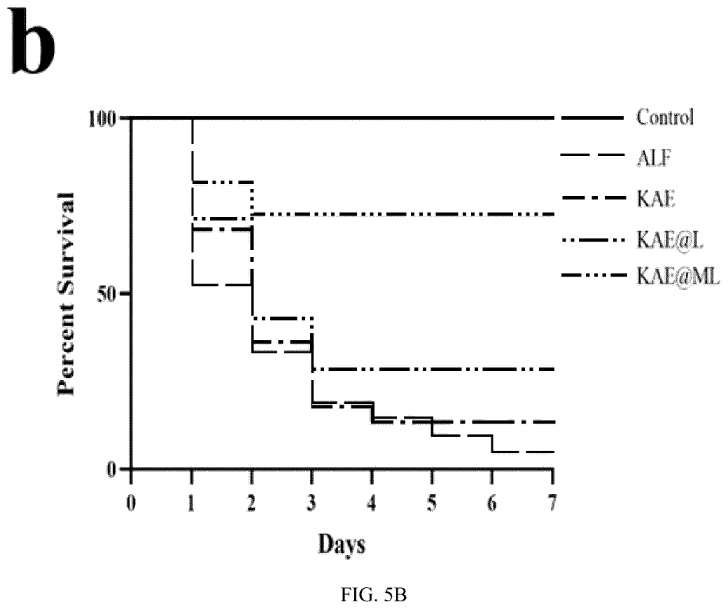

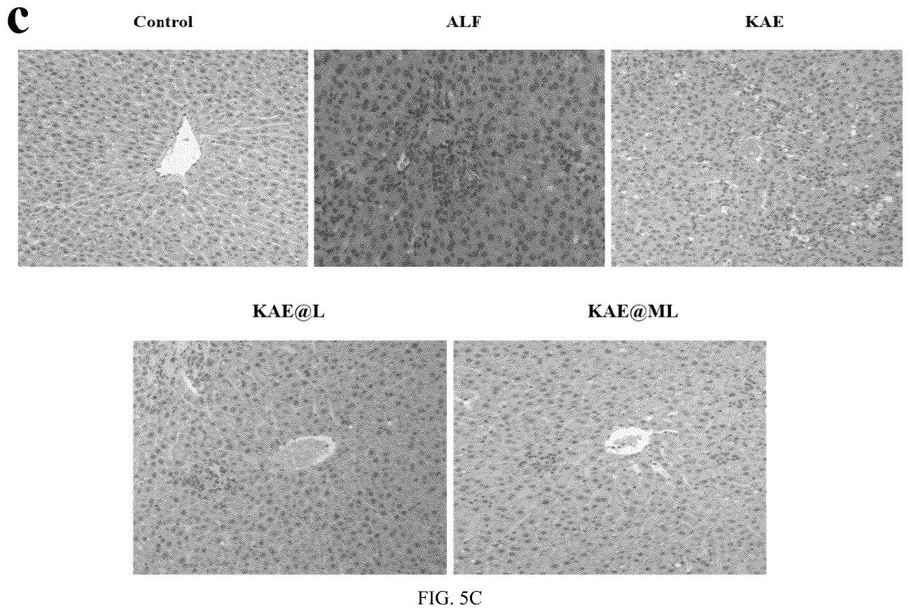

The liver function levels of the SD rats in each treatment group were further detected using an automatic analyzer, and the results showed an increase in the liver function level in the rats after treatment. The aspartate aminotransferase (AST) value was 213.15±4.00(U/L) in the rats of the control group, 1492.33±21.01(U/L) in the rats of the ALF group, 824.24±23.54(U/L) in the rats of the KAE group, 786.02±12.79 (U/L) in the rats of the KAE@L group, and 579.83±12.27(U/L) in the rats of the KAE@ML group. During alanine aminotransferase (ALT) detection, the values of the control group, the ALF group, the KAE group, the KAE@L group, the KAE group, and the KAE@ML group were 47.25±3.40(U/L), 1058.34±18(U/L), 791.71±14.03(U/L), 627.70±13.8(U/L), and 444.38±19.41(U/L), respectively, (FIG. 5A). Therefore, the AST and ALT levels in the rats were somewhat reduced in the KAE group, the KAE@L group and the KAE@ML group, and significantly reduced in the KAE@ML group which showed alleviated liver inflammation, as compared with the ALF group. The survival graph showed that compared with other groups, the KAE@ML group exhibited significantly increased survival rate of the rats (FIG. 5B). HE (hematoxylin-eosin staining) results also indicated that the liver injury of the rats was significantly alleviated after treatment with KAE@ML, as compared with other groups (FIG. 5C). These indicated that KAE@ML had a good therapeutic effect on the LAF rats.

The embodiments described above are merely preferred embodiments of the present invention and not intended to limit the present invention. Any of modifications, equivalent substitutions, improvements, etc. made within the spirit and principle of the present invention shall be covered in the scope of the present invention.

What is claimed is:

1. A kaempferol biomimetic nano-material, comprising liposomes for mesenchymal stem cell membranes and kaempferol liposomes.

2. The kaempferol biomimetic nano-material according to claim 1, wherein the kaempferol biomimetic nano-material has an average particle size of 135-140 nm.

3. The kaempferol biomimetic nano-material according to claim 1, wherein a method for preparing the kaempferol biomimetic nano-material comprises steps of:
1) preparing the mesenchymal stem cell membranes: culturing and collecting mesenchymal stem cells, sequentially performing a first centrifuging, a first resuspending, a second centrifuging, a second resuspending, a freezing-thawing, and a third centrifuging on the mesenchymal stem cells, and finally collecting cell pellets, wherein the cell pellets are the mesenchymal stem cell membranes;
2) preparing the kaempferol liposomes: dissolving lecithin, cholesterol, and kaempferol in absolute ethanol to obtain a first mixture, adding the first mixture to ultrapure water to obtain a second mixture, stirring the second mixture to obtain a stirred mixture, and extruding the stirred mixture using a liposome extruder to pass through films to obtain the kaempferol liposomes; and
3) extruding the mesenchymal stem cell membranes obtained in the step 1) using the liposome extruder to obtain extruded mesenchymal stem cell membranes, mixing the extruded mesenchymal stem cell membranes with the kaempferol liposomes to obtain a third mixture, ultrasonically shaking the third mixture in an ice bath to obtain a shaken mixture, and extruding the shaken mixture using the liposome extruder to obtain the kaempferol biomimetic nano-material.

4. The kaempferol biomimetic nano-material according to claim 3, wherein in the step 1), the first resuspending is to resuspend first centrifuged cells in a phosphate buffer saline, and the second resuspending is to resuspend second centrifuged cells in a hypotonic solution.

5. The kaempferol biomimetic nano-material according to claim 4, wherein the hypotonic solution is an aqueous solution containing 10 mmol/L tris (hydroxymethyl)-aminomethane hydrochloride (Tris-HCL), 1 mmol/L KCl, 1.5 mmol/L MgCl₂, and 1 mmol/L phenylmethanesulfonyl fluoride (PMSF) by concentration content, respectively.

6. The kaempferol biomimetic nano-material according to claim 3, wherein in the step 2), a mass ratio of the lecithin to the cholesterol to the kaempferol is 3:1:1.

7. The kaempferol biomimetic nano-material according to claim 3, wherein in the step 3), a mass ratio of the extruded mesenchymal stem cell membranes to the kaempferol liposomes is 1:1-1:10.

8. The kaempferol biomimetic nano-material according to claim 3, wherein in the step 1), the first centrifuging, the second centrifuging, or the third centrifuging is carried out at 4° C.

9. The kaempferol biomimetic nano-material according to claim 3, wherein in the step 2), the kaempferol liposomes are obtained by sequential extruding through the films of 800 nm, 400 nm, and 200 nm on the liposome extruder, and the films are polycarbonate films.

10. A use of the kaempferol biomimetic nano-material according to claim 1 in a preparation of a drug for a treatment of an acute hepatic failure.

11. The kaempferol biomimetic nano-material according to claim 2, wherein a method for preparing the kaempferol biomimetic nano-material comprises steps of:
1) preparing the mesenchymal stem cell membranes: culturing and collecting mesenchymal stem cells, sequentially performing a first centrifuging, a first resuspending, a second centrifuging, a second resuspending, a freezing-thawing, and a third centrifuging on the mesenchymal stem cells, and finally collecting cell pellets, wherein the cell pellets are the mesenchymal stem cell membranes;
2) preparing the kaempferol liposomes: dissolving lecithin, cholesterol, and kaempferol in absolute ethanol to obtain a first mixture, adding the first mixture to ultrapure water to obtain a second mixture, stirring the second mixture to obtain a stirred mixture, and extruding the stirred mixture using a liposome extruder to pass through films to obtain the kaempferol liposomes; and
3) extruding the mesenchymal stem cell membranes obtained in the step 1) using the liposome extruder to obtain extruded mesenchymal stem cell membranes, mixing the extruded mesenchymal stem cell membranes with the kaempferol liposomes to obtain a third mixture, ultrasonically shaking the third mixture in an ice bath to obtain a shaken mixture, and extruding the shaken mixture using the liposome extruder to obtain the kaempferol biomimetic nano-material.

12. The kaempferol biomimetic nano-material according to claim 11, wherein in the step 1), the first resuspending is to resuspend first centrifuged cells in a phosphate buffer saline, and the second resuspending is to resuspend second centrifuged cells in a hypotonic solution.

13. The use according to claim 10, wherein the kaempferol biomimetic nano-material has an average particle size of 135-140 nm.

14. The use according to claim 10, wherein a method for preparing the kaempferol biomimetic nano-material comprises steps of:
1) preparing the mesenchymal stem cell membranes: culturing and collecting mesenchymal stem cells, sequentially performing a first centrifuging, a first resuspending, a second centrifuging, a second resuspending, a freezing-thawing, and a third centrifuging on the mesenchymal stem cells, and finally collecting cell pellets, wherein the cell pellets are the mesenchymal stem cell membranes;
2) preparing the kaempferol liposomes: dissolving lecithin, cholesterol, and kaempferol in absolute ethanol to obtain a first mixture, adding the first mixture to ultrapure water to obtain a second mixture, stirring the second mixture to obtain a stirred mixture, and extruding the stirred mixture using a liposome extruder to pass through films to obtain the kaempferol liposomes; and
3) extruding the mesenchymal stem cell membranes obtained in the step 1) using the liposome extruder to obtain extruded mesenchymal stem cell membranes, mixing the extruded mesenchymal stem cell membranes with the kaempferol liposomes to obtain a third mixture, ultrasonically shaking the third mixture in an ice bath to obtain a shaken mixture, and extruding the shaken mixture using the liposome extruder to obtain the kaempferol biomimetic nano-material.

15. The use according to claim 14, wherein in the step 1) of the method for preparing the kaempferol biomimetic nano-material, the first resuspending is to resuspend first centrifuged cells in a phosphate buffer saline, and the second resuspending is to resuspend second centrifuged cells in a hypotonic solution.

16. The use according to claim 15, wherein in the method for preparing the kaempferol biomimetic nano-material, the hypotonic solution is an aqueous solution containing 10 mmol/L Tris-HCL, 1 mmol/L KCl, 1.5 mmol/L MgCl₂, and 1 mmol/L PMSF by concentration content, respectively.

17. The use according to claim 14, wherein in the step 2) of the method for preparing the kaempferol biomimetic nano-material, a mass ratio of the lecithin to the cholesterol to the kaempferol is 3:1:1.

18. The use according to claim 14, wherein in the step 3) of the method for preparing the kaempferol biomimetic nano-material, a mass ratio of the extruded mesenchymal stem cell membranes to the kaempferol liposomes is 1:1-1:10.

19. The use according to claim 14, wherein in the step 1) of the method for preparing the kaempferol biomimetic nano-material, the first centrifuging, the second centrifuging, or the third centrifuging is carried out at 4° C.

20. The use according to claim 14, wherein in the step 2) of the method for preparing the kaempferol biomimetic nano-material, the kaempferol liposomes are obtained by sequential extruding through the films of 800 nm, 400 nm, and 200 nm on the liposome extruder, and the films are polycarbonate films.
